(19) United States
(12) Patent Application Publication
(10) Pub. No.: US 2024/0005760 A1
(43) Pub. Date: Jan. 4, 2024

(54) A NURSE COMMUNICATION DEVICE

(71) Applicant: Kush PRAJAPATI, Naranpura, Ahmedabad (IN)

(72) Inventor: Kush PRAJAPATI, Naranpura, Ahmedabad (IN)

(21) Appl. No.: 18/253,787

(22) PCT Filed: Nov. 20, 2021
(86) PCT No.: PCT/IB2021/060774

§ 371 Date: May 22, 2023

(30) Foreign Application Priority Data
Nov. 20, 2020 (IN) 202021050574

(51) Int. Cl. G08B 21/02; G16H 40/63

(52) U.S. Cl. CPC G08B 21/0211; G16H 40/63

(57) ABSTRACT

The present invention relates to a nurse communication device (100). The nurse communication device (100) comprising patient monitoring unit and nurse calling button (101). The patient monitoring device and the nurse calling device both having indicator assembly. The individual nurse having individual RFID card which having all the personal information of the individuals. The nurse communication device (100) synchronous with the cloud through the router. The nurse communication device (100) easily paired with the voice assistant devices like siri or Alexa. The nurse calling device can provide facility to access real time data through the use of login credential. The application of the nurse communication device (100) generate accurate calling report consisting all the information like when patient called for nurse, who attended and at what time etc.

### FIELD OF THE INVENTION

The present invention relates to a nurse communication device. More particularly the present invention relates to a patient care and communication system which adequate to executing tasks such as visual and data communications and tracking the location of staff members to provide maximum patient care.

### BACKGROUND OF THE INVENTION

Nurses and other caretakers in a hospital must work under varying conditions. These caretakers or nurses must remain alert to respond to patient needs, in emergency and nonemergency situations. Due to economic practicalities and the ever-increasing costs of medical care, it is necessary to make the most efficient use of nurses and staff on call in a hospital wing, particularly at night when nurse and staff levels are maintained at a minimum.

On the other hand, a desire to optimize the efficiency of nurse and caretaker is of secondary importance relative to providing a high level of medical care. If nurse and staff levels are reduced to reduce costs without corresponding opportunities to improve efficiency, the level of patient care will decrease. One approach to maximizing the efficiency of nurses and other hospital staff involves the use of a location and identification system to continuously monitor the locations of these persons.

To more fully understand the above problem relating to health care, consider the types of data which are maintained for an individual patient. As such hospitals needed to track the record of the treatments provided to the patient. To understand deeply, it is needed to maintain the data as such which nurse attends the patient at what time, etc.

Presently known systems manage such information includes manual writing and processing of the information. Electronic systems utilized to process and store the information involve multiple computers, each configured to process portions of the vast amount of information. To obtain all the information in one place the information stored in each computer system must be combined manually. Furthermore, such electronic systems do not provide visual displays of text at stations provided in the patient's room, at the nurse control station, or stations provided in areas of the health care facility frequently occupied by the health care personnel.

Further, in the large hospitals numbers of patients admitted. It is quite difficult to maintain all the patient regularly without any interferences. Recent technologies provides the bell button near the bed of the patient so can the nurse can come and receive the patient. But it is very difficult to understand that if the nurse attend the patient or not. As such it is possible that more than two nurses can reach to attend a single patient. Due to this the confusions created between the staff members that weather the patient attended by any of them or not.

Various prior art discloses about the communication device for patient and hospital staff. The prior art document U.S. Pat. No. 5,822,544A discloses a patient care and communication system which utilizes a central processing system and a plurality of remote stations electrically connected to the central processing system to facilitate audio, visual and data communications. The central processing system facilitates the audio, visual and data communications between the plurality of remote stations, and includes a system for determining which of the plurality of remote stations are transmitting the audio, visual and data communications and which of the plurality of remote stations are to receive the audio, visual and data communications. The central processing system also includes a system which establishes a communication link between the transmitting stations and the receiving stations. The remote stations include a processing system which also facilitates the audio, visual and data communications and a display for displaying the visual communications.

Another prior art document U.S. Pat. No. 5,838,223A discloses a patient/nurse call system for a hospital includes patient stations capable of generating hospital calls and a remote master station which prioritizes and stores the calls. Hall units outside patient rooms identify the rooms from which the calls originate and the type of call. Each patient station allows selective retrieval and display of unanswered calls, and the system further permits audio interconnection between any two of the stations. Nurse-worn badges transmit pulse-coded infrared signals which are received by receivers at the patient stations and in response, the systems generates identity and location signals which are stored at the master station. The nurse location and identity information may also be retrieved and displayed at any patient station.

Another prior art document EP1865833B2 disclosed the location detection system comprises a first locating device associated with the patient handling device for transmitting a first unique location identifier to the processing station and a second locating device associated with the patient handing device for transmitting a second unique location identifier to the processing station. In this system, the first unique location identifier corresponds to a first area of the location and the second unique location identifier corresponds to a second area of the location different than the first area. In one embodiment, tine first unique location identifier identifies the room in which the patient handling device is located and the second unique location identifier identifies the zone in the room in which the patient handling device is positioned.

Another prior art document U.S. Pat. No. 10,098,593B2 disclosed the system that monitors various conditions of a plurality of hospital beds located in different rooms of a healthcare facility is provided. Alternatively or additionally, other types of equipment may be monitored by the system. Various configurations of network interface units that are coupleable to or integrated into a hospital bed are also disclosed. The system receives data from the hospital beds and/or other equipment and initiates a communication to a wireless communication device of at least one designated caregiver in response to the received data being indicative of an alarm condition.

The aforementioned prior arts encounter various problems which lead into the difficulties in many ways. However, there have been many attempts to provide a communication and patient care device, but those known applications have certain disadvantages like lack of real time monitoring, lack of information, lack of automation, lack of safety of the patients in case of emergency, etc.

As discussed above, all the known devices having one or more problem like, manual operation, time consuming, and database management. Hence, it is desperately needed to introduce a nurse communication device which overcomes all mentioned problems.

### OBJECT OF THE INVENTION

The principle object of the present invention is to provide a nurse communication device.

Another object of the present invention is to provide a nurse communication device which connected to the cloud so can anyone can watch real time data of the status of the nurse calling device.

Another object of the present invention is to provide a nurse communication device which provide the feature for the audio and video call from the nurse calling device.

Another object of the present invention is to provide a nurse communication device which can easily pared with other voice assistance devices like siri, alexa, etc.

Another object of the present invention is to provide a nurse communication device which can have a capacitive touch button so that patient can easily access it.

Another object of the present invention is to provide a nurse communication device which works on the rechargeable battery having higher battery life and having battery level indicator.

Another object of the present invention is to provide a nurse communication device which generating accurate calling report including all necessary information.

Another object of the present invention is to provide location tracking facility of individuals.

### SUMMARY OF THE INVENTION

This summary is provided to introduce a selection of concepts in a simplified form that are further disclosed in the detailed description of the invention. This summary is not intended to identify key or essential inventive concepts of the claimed subject matter, nor is it intended for determining the scope of the claimed subject matter.

The present invention is all about a patient care and communication system which adequate to executing tasks such as visual and data communications and tracking the location of staff members to provide maximum patient care.

The main aspect of the present invention is to provide a nurse communication device comprising a patient monitoring unit having means for monitoring and storing patient data and for determining the location of individuals within a health care facility; at least one nurse calling button, positioned on one of a plurality of patient bed, said nurse calling button being coupled to said patient monitoring unit and having a processor unit which facilitates data signal communications with said central unit; a main circuit unit being capable to provide the operational power to all peripheral units; a side panel having temperature sensor being capable to sense the temperature of the patient's body as well as the room temperature; a charging port and a On/off button for operating the nurse communication device; a fingerprint lock sensor being capable to lock and unlock the nurse communication device for the security purposes; a display unit being capable to show the functions on the screen; and at least one indicator assembly having buzzer for indicating the presence of the individual personnel in said one of a plurality of patient rooms and in said central unit, coupled to said at least one nurse calling button such that when said patient monitoring unit determines the location of personnel in said one of a plurality of patient rooms, said patient monitoring unit transfers actuation signals to said at least one nurse communication device to actuate said indicator assembly which indicates current situation wherein, said nurse communication device operated through a plurality of RFID cards, each configured for attachment to individual and having a database for storing information including identity information about the individual personnel to which said respective card is equipped, and configured to transmit of said information to one of a plurality of RFID sensors to access the information of individual personnel information for the analysis, said plurality of RFID sensors being synchronous to said patient monitoring unit to transfer signals representing information received from said RFID card to permit said patient monitoring unit to determine the location of individual.

One more aspect of the present invention is to provide patient monitoring unit having a display as an indicator assembly consisting of three indication LED for the different situations; a data storage and retrieval unit to upload said data to cloud and restore it whenever required.

One more aspect of the present invention is to provide indicator assembly includes a plurality of indicators on said nurse calling device and said central unit, and said patient monitoring unit determines from said identity information the location and information of personnel, such that when said patient monitoring unit determines the presence of personnel in said predetermined location said patient monitoring unit transfers to said nurse calling device actuation signals to actuate one of said plurality of indicators associated with said personnel.

Yet another aspect of the present invention is to provide the nurse calling device which suitable to work with voice assistance; voice and video calls and work on the internet of things.

One another aspect of the present invention is to provide nurse calling device having plurality of buttons for calling, code blue and reset; said RFID sensor to access the information of individual personnel information for the analysis.

### BRIEF DESCRIPTION OF THE DRAWINGS

The foregoing summary, as well as the following detailed description of the invention, is better understood when read in conjunction with the appended drawings. For the purpose of illustrating the invention, exemplary constructions of the invention are shown in the drawings.

FIG. 1 shows schematic diagram of nurse communication device of the present invention.

FIG. 2 shows the schematic diagram of the side panel and front panel of the present invention.

### DETAILED DESCRIPTION OF INVENTION

Detailed embodiments of the present invention are disclosed herein, however, it is to be understood that the disclosed embodiments are merely exemplary of the invention, which may be embodied in various forms. Therefore, specific functional and structural details disclosed herein are not to be interpreted as limiting, but merely as a basis for the claims and as a representative basis for teaching one skilled in the art to variously employ the present invention in virtually any appropriately detailed structure.

Unless defined otherwise, all technical and scientific terms used herein have the same meaning as commonly understood by one of ordinary skill in the art to which the invention belongs.

The present invention overcomes the aforesaid drawbacks of the above and other objects, features and advantages of the present invention will now be described in greater detail. Also, the following description includes various specific details and is to be regarded as merely exemplary. Accordingly, those of ordinary skill in the art will recognize that: without departing from the scope and spirit of the present disclosure and its various embodiments there may be any number of changes and modifications described herein.

It must also be noted that as used herein and in the appended claims, the singular forms “a”, “an,” and “the” include plural references unless the context clearly dictates otherwise. Although any systems and methods similar or equivalent to those described herein can be used in the practice or testing of embodiments of the present invention, the preferred, systems and methods are now described.

The present invention is all about a nurse communication device which adequate to executing tasks such as visual and data communications and tracking the location of staff members to provide maximum patient care.

As per main embodiment of the present invention, a nurse communication device comprising a patient monitoring unit (100) having means for monitoring and storing patient data and for determining the location of individuals within a health care facility; at least one nurse calling button (101), positioned on one of a plurality of patient bed, said nurse calling button (101) being coupled to said patient monitoring unit (100) and having a processor unit which facilitates data signal communications with said central unit; a main circuit unit (105) being capable to provide the operational power to all peripheral units; a side panel having temperature sensor (110) being capable to sense the temperature of the patient's body as well as the room temperature; a charging port (111) and a On/off button (112)for operating the nurse communication device; a fingerprint lock sensor (113) being capable to lock and unlock the nurse communication device for the security purposes; a display unit (106) being capable to show the functions on the screen; and at least one indicator assembly having buzzer (108) for indicating the presence of the individual personnel in said one of a plurality of patient rooms and in said central unit, coupled to said at least one nurse calling button such that when said patient monitoring unit determines the location of personnel in said one of a plurality of patient rooms, said patient monitoring unit transfers actuation signals to said at least one nurse communication device to actuate said indicator assembly which indicates current situation wherein, said nurse communication device operated through a plurality of RFID cards, each configured for attachment to individual and having a database for storing information including identity information about the individual personnel to which said respective card is equipped, and configured to transmit of said information to one of a plurality of RFID sensors (102) to access the information of individual personnel information for the analysis, said plurality of RFID sensors (102) being synchronous to said patient monitoring unit to transfer signals representing information received from said RFID card to permit said patient monitoring unit to determine the location of individual.

As used herein, “RFID” means Radio-frequency identification (RFID) which uses electromagnetic fields to automatically identify and track tags attached to respective objects. An RFID tag consists of a tiny radio transponder; a radio receiver and transmitter. When triggered by an electromagnetic interrogation pulse from a nearby RFID reader device, the tag transmits digital data, usually an identifying inventory number, back to the reader. This number can be used to track inventory goods.

As per another embodiment of the present invention the patient monitoring unit having a display as an indicator assembly consisting of three indication LED for the different situations; a data storage and retrieval unit to upload said data to cloud and restore it whenever required. The indication LED are red, yellow and green. The red indication LED indicating that Patient Calling for Nurse. This indicator actuate only when patient pressed the nurse calling button. The yellow indication LED indicates that the call is accepted and now nurse is going to attend patient. Due to this indication, other staff member also get aware about that the patient will attending by someone. The green indication LED indicates that the nurse has attended the patient. In case of code blue, big font indicating the emergency condition and send data to associated devices like other phone or display which handled by doctor or ambulance.

As per another embodiment of the present invention the nurse communication device being connected with smart watch for monitoring the patient's data such as heart rate, blood pressure, oxygen level, stress level, emotional condition, ECG, real-time hand movement, walking activity, sleeping pattern and fall detection.

As per another embodiment of the present invention the nurse calling device having plurality of buttons. When the patient need for the nurse, the patient pressed the nurse calling button (101). Due to this, the signal through the router passed to the patient monitoring room and indicating red light on both i.e. in patient monitoring room and on nurse calling device. The nurse communication device also having RFID sensor through which the detailed information of the nurse attendance transmitted to the patient monitoring system and also uploaded on the cloud. The code blue button (103) provided for the emergency situation when the patient needs urgent treatment. The code blue button (103) can be pressed after the nurse tap the RFID card on the sensor (102). The reset buttons (104) provided separately for the calling button and code blur button (103). Also the nurse communication device having two LED indication. The red LED indicating calling and green LED indicating nurse received call.

As per another embodiment of the present invention the indicator assembly is deactuated by said at least one nurse calling device when said patient monitoring unit determines that the nurse fulfill all the needs of the patient and after tapping the RFID card left the patient room.

As per another embodiment of the present invention is that indicator assembly includes a plurality of indicators on said nurse calling device and said patient monitoring unit, and said patient monitoring unit determines from said identity information the location and information of personnel, such that when said patient monitoring unit determines the presence of personnel in said predetermined location said patient monitoring unit transfers to said nurse calling device actuation signals to actuate one of said plurality of indicators associated with said personnel.

As per another embodiment of the present invention the display (106) further shows connectivity status, battery status, real-time patients data such as oxygen level, heart rate, blood pressure, hand and walk movement, ECG, stress level, emotional condition, calling facility, nurses data, doctor visit data, medication data and sleeping pattern.

As per another embodiment of the present invention the mobile application can generate accurate calling report. The accurate calling report consist of when patient called for nurse, who attended and at what time, code blue duration and number of times, which doctor/caretaker attended patient over the time of treatment process, how many times delayed occurred in attending patient, accurate data analysis report of nurse calling task with number and charts.

As per another embodiment of the present invention the nurse communication device suitable to work with voice assistance; voice and video calls and work on the internet of things. The nurse communication device (100) also provide secure connection to the router so can anyone from anywhere through the login credential access the real time data.

As per another embodiment of the present invention when the patient call generated from the nurse communication device (100), the red LED start indicating on the nurse communication device (100). During the same time a signal also transmitted to the patient monitoring unit where the red LED start blinking. The nurse available at the patient monitoring unit received a call and the red LED stops blinking. When the nurse received the call the indicator assembly shows yellow LED which indicates that the nurse has accepted the call and on the way to attend the patient. Due to this indication, the other staff member also going to know that someone attending the patient and chance of confusion became zero. The nurse after reached to the patient's room, tap the RFID card on the RFID sensor (102) mounted at the nurse communication device (100) so that the information of the nurse transmitted automatically to the system as well as on the cloud. In case of the emergency, the nurse after tapping RFID card on the sensor (102) can access the code blue button (103). During the time of code blue, all features like Audio and video call activated. So nurses/doctors can immediately connect with patients. Also during a code blue, all authorities get the real-time alarm on their smart watch, phone, or computer/laptop screen. The nurse communication device (100) having reset key for both calling and code blue button (103). So once the task completed the situation can be reset by pressing the respective reset button and after the nurse tapping the RFID card again, the indicator assembly of the patient monitoring unit turned yellow and then deactuated it means the nurse was attended the patient.

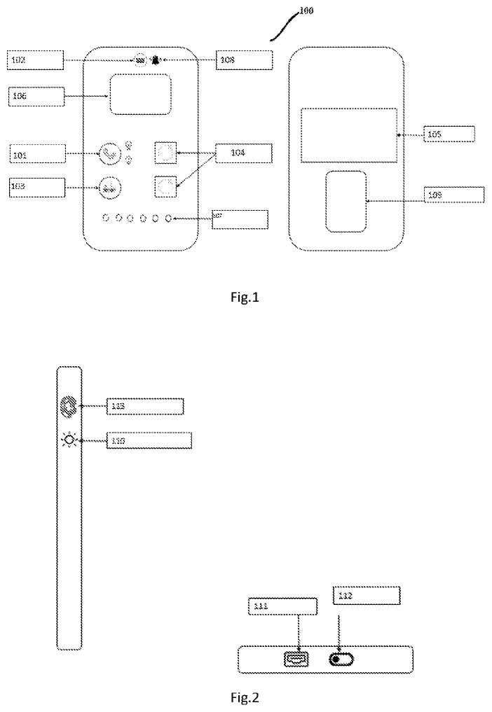

Referring to FIG. 1, it shows the nurse communication device (100) being connected constantly with the patient monitor unit. The patient monitoring unit being capable to store and monitor the patient data. The nurse calling button (101) provided on the nurse communication device (101) which provides the feature to call the nurse at any time. The indicator assembly having buzzer (108) for indicating the presence of the individual personnel in said one of a plurality of patient rooms and in said central unit, coupled to said at least one nurse calling button such that when said patient monitoring unit determines the location of personnel in said one of a plurality of patient rooms. The patient monitoring unit transfers actuation signals to said at least one nurse communication device to actuate said indicator assembly which indicates current situation. The nurse communication device operated through a plurality of RFID cards, each configured for attachment to individual and having a database for storing information including identity information about the individual personnel to which said respective card is equipped, and configured to transmit of said information to one of a plurality of RFID sensors (102) to access the information of individual personnel information for the analysis, said plurality of RFID sensors (102) being synchronous to said patient monitoring unit to transfer signals representing information received from said RFID card to permit said patient monitoring unit to determine the location of individual.

Without further description, it is believed that one of ordinary skill in the art can, using the preceding description and the illustrative examples, make and utilize the present invention and practice the claimed methods. It should be understood that the foregoing discussion and examples merely present a detailed description of certain preferred embodiments. It will be apparent to those of ordinary skill in the art that various modifications and equivalents can be made without departing from the spirit and scope of the invention.

### LIST OF REFERENCE NUMERALS

Nurse communication device (100)

Nurse calling button (101)

Main circuit unit (105)

Temperature sensor (110)

Charging port (111)

On/off button (112)

Fingerprint lock sensor (113)

Display unit (106)

Buzzer (108)

RFID sensors (102)

Code blue (103), reset (104)

Electronic appliances (107)

Display (106)

Rechargeable battery (109)

1. A nurse communication device comprising:
a patient monitoring unit having means for monitoring and storing patient data and for determining the location of individuals within a health care facility;
at least one nurse calling button, positioned on one of a majority of patient bed, said nurse calling button being coupled to said patient monitoring unit and having a processor unit which facilitates data signal communications with said central unit;
a main circuit unit being capable of providing the operational power to all peripheral units;
a side panel having temperature sensor being able to sense the temperature of the patient's body as well as the room temperature;
a charging port and an On/off button for operating the nurse communication device;
a fingerprint lock sensor being able to lock and unlock the nurse communication device for the security purposes;
a display unit being able to show the functions on the screen; and
at least one indicator assembly having buzzer for indicating the presence of the individual personnel in said one of a plurality of patient rooms and in said central unit, coupled to said at least one nurse calling button such that when said patient monitoring unit determines the location of personnel in said one of a plurality of patient rooms, said patient monitoring unit transfers actuation signals to said at least one nurse communication device to actuate said indicator assembly which indicates current situation;
wherein, said nurse communication device operated through a plurality of RFID cards, each configured for attachment to individual and having a database for storing information including identity information about the individual personnel to which said respective card is equipped, and configured to transmit of said information to one of a plurality of RFID sensors to access the information of individual personnel information for the analysis, said majority of RFID sensors being synchronous to said patient monitoring unit to transfer signals representing information received from said RFID card to permit said patient monitoring unit to determine the location of individual.

2. The nurse communication device as claimed in claim 1, wherein said patient monitoring unit having a display as an indicator assembly consisting of three indication LEDs for the different situations; a data storage and retrieval unit to upload said data to cloud and restore it whenever required.

3. The nurse communication device as claimed in claim 1, wherein said nurse communication device having plurality of buttons for calling, code blue, reset and buttons for controlling the peripheral electronic appliances.

4. The nurse communication device as claimed in claim 1, wherein indicator assembly is deactuated by said at least one nurse communication device when said patient monitoring unit determines that the personnel has left said predetermined location.

5. The nurse communication device as claimed in claim 1, wherein indicator assembly includes a plurality of indicators on said nurse calling device and said patient monitoring unit, and said patient monitoring unit determines from said identity information the location and information of personnel, such that when said patient monitoring unit determines the presence of personnel in said predetermined location said patient monitoring unit transfers to said nurse communication device actuation signals to actuate one of said plurality of indicators associated with said personnel.

6. The nurse communication device as claimed in claim 1, wherein said nurse communication device suitable to work with voice assistance; voice and video calls and work on the internet of things.

7. The nurse communication device as claimed in claim 1, wherein said nurse communication device being connected with smart watch for monitoring the patient's data such as heart rate, blood pressure, oxygen level, stress level, emotional condition, ECG, Realtime hand movement, walking activity, sleeping pattern and fall detection.

8. The nurse communication device as claimed in claim 1, wherein display further shows connectivity status, battery status, realtime patients data such as oxygen level, heart rate, blood pressure, hand and walk movement, ECG, stress level, emotional condition, calling facility, nurses data, doctor visit data, medication data and sleeping pattern.

9. The nurse communication device as claimed in claim 1, wherein said nurse communication device having a rechargeable battery making the device rechargeable and portable.
